(19) United States
(12) Patent Application Publication
(10) Pub. No.: US 2025/0249154 A1
(43) Pub. Date: Aug. 7, 2025

(54) TORTUOUS PATH FILTER FOR THE PRODUCTION OF TRANSFUSION-READY RECONSTITUTED PLASMA

(71) Applicant: Hemerus Medical, LLC, St. Paul, MN (US)

(72) Inventors: Majid Zia, St. Paul, MN (US); Nancy C Wilker, Lexington, MA (US)

(73) Assignee: Hemerus Medical, LLC, St. Paul, MN (US)

(21) Appl. No.: 19/189,001

(22) Filed: Apr. 24, 2025

Related U.S. Application Data
(63) Continuation of application No. PCT/US2023/036332, filed on Oct. 31, 2023.
(60) Provisional application No. 63/431,329, filed on Dec. 9, 2022.

(51) Int. Cl. A61M 1/02; B01D 29/01

(52) U.S. Cl. CPC A61M 1/0281; B01D 29/018; A61M 2202/0028; A61M 2202/0415; B01D 2201/184

(57) ABSTRACT

A tortuous path filter for filtering reconstituted plasma, the filter comprising at least two layers of fibers, single-layer filters, or beads, wherein the filter lacks or substantially lacks pores defining a lumen, said lumen having a diameter of larger than (100) microns along a straight path through the filter. The invention provides devices including a tortuous path filter, and methods for producing transfusion-ready plasma comprising: (a) reconstituting dried plasma to produce reconstituted plasma; and (b) passing said reconstituted plasma through the tortuous path filter to produce transfusion-ready plasma.

### CROSS-REFERENCE TO RELATED APPLICATIONS

This application claims priority benefit from U.S. Provisional Application No. 63/431,329, filed Nov. 1, 2022, the entire contents of which is hereby incorporated by reference.

### BACKGROUND

The invention relates to the field of medicine and surgery and, more specifically, to the field of transfusion medicine.

Whole blood consists of a cellular component (e.g., white blood cells, red blood cells, and platelets) suspended in a liquid called plasma. Plasma makes up more than 50% of the overall content of blood and, when separated from the cellular component, presents as a liquid with a light yellow color.

Plasma, separated from the cellular components of whole blood, is often transfused to patients suffering from a traumatic injury to control bleeding and to alleviate any coagulopathy by restoring hemostasis. While traumatic injury is the leading cause of death for patients under 35 years of age and is responsible overall for approximately 10% of all deaths worldwide, survival from hemorrhage can be improved when treatment efforts include administering plasma (by transfusion) within the first six hours following injury.

Currently, plasma products, such as fresh frozen plasma (FFP), are stored frozen and thawed within 24 hours prior to transfusion to a patient in need. However, in settings where trauma is likely to occur (e.g., a battlefield), the absence of a freezer and/or thawer delays the ability to administer plasma quickly to patients in need. Rapid administration of plasma to a patient in need is important. In a recent study in warfarin-related intracranial hemorrhage, each 30 minute delay in administering the first dose of plasma translated into a 20% lower chance of reversing the patient's coagulopathy within the first 24 hours.

Given that the major component of plasma is water, efforts were made to produce a dried plasma product that could be stored at ambient temperatures and reconstituted when needed for transfusion. Dried plasma products were used by the armed forces during World War II, particularly to maintain blood pressure and prevent shock in soldiers who had experienced large volumes of blood loss. However, because the dried plasma products used during World War II were pooled from multiple donors, contamination with viral pathogens (e.g., hepatitis) sometimes occurred, and the continued use of dried plasma product for transfusion to human patients in need fell out of favor in the 1950's.

Recently, improved methods to ensure blood and blood product safely has led to a renewed interest in dried plasma products, and reconstituted dried plasma for patient transfusions. However, safety and other logistical concerns remain. For example, the United States has not resumed the use of reconstituted dried plasma products in patient transfusions.

There is a need, therefore, for transfusion-ready reconstituted plasma, and methods and devices for producing the same.

### SUMMARY

In some embodiments, the invention provides transfusion-ready reconstituted plasma, and methods and devices for producing the same.

In a first aspect, the invention provides tortuous path filter for filtering reconstituted plasma, the filter comprising at least two layers of fibers, single-layer filters, or beads, wherein the filter lacks or substantially lacks pores defining a lumen having a diameter larger than 100 microns along a straight path through the filter.

In another aspect, the invention provides a medical device comprising a tortuous path filter of for filtering reconstituted plasma, the filter comprising at least two layers of fibers, single-layer filters, or beads, wherein the filter lacks or substantially lacks pores defining a lumen having a diameter larger than 100 microns along a straight path through the filter, said filter inline with a first tubing to an inlet port and a second tubing to an outlet port, wherein said inlet port further comprises a spike and said outlet port further comprising a needle for intravenous injection.

In another aspect, the invention provides a blood collection system comprising a tortuous path filter, said tortuous path filter comprising at least two layers of fibers, single-layer filters, or beads, wherein the filter lacks or substantially lacks pores defining a lumen having a diameter larger than 100 microns along a straight path through the filter, the filter component inline with a first tubing to an inlet port and a second tubing to an outlet port, wherein said inlet port further comprises a spike and said outlet port further comprising a needle for intravenous injection and a blood bag comprising a port injectable with the spike of the tortuous path filter. In some embodiments, the blood bag comprises a blood bag with an internal surface, said internal surface coated with one or more blood group antigens.

In yet another aspect, the invention provides a method for producing transfusion-ready plasma comprising: (a) reconstituting dried plasma (e.g., freeze dried plasma or spray dried plasma) to produce reconstituted plasma; and (b) passing said reconstituted plasma through a tortuous path filter comprising at least two layers of fibers, single-layer filters, or beads, wherein the filter lacks or substantially lacks pores defining a lumen having a diameter larger than 100 microns along a straight path through the filter, to produce transfusion-ready plasma. In some embodiments, the filter meets a standard selected from the group consisting of a United States Pharmacopeia (USP) 788 standard and American National Standards Institute (ANSI)/Association for the Advancement of Medical Instrumentation (AAMI) BF7 standard.

In another aspect, the invention provides transfusion-ready plasma produced by (a) reconstituting dried plasma (e.g., freeze dried plasma or spray dried plasma) to produce reconstituted plasma; and (b) passing said reconstituted plasma through the tortuous path filter comprising at least two layers of fibers, single-layer filters, or beads, wherein the filter lacks or substantially lacks pores defining a lumen having a diameter larger than 100 microns along a straight path through the filter. In some embodiments, the filter meets a standard selected from the group consisting of a United States Pharmacopeia (USP) 788 standard and American National Standards Institute (ANSI)/Association for the Advancement of Medical Instrumentation (AAMI) BF7 standard. In various embodiments, the transfusion-ready plasma lacks or substantially lacks a transfusion-preventing particle. For example, in various embodiments, the transfusion-ready plasma lacks or substantially lacks a transfusion-preventing particle comprising an aspect ratio of length to width of at least 10, and wherein the particle is at least 40 microns in length. In various embodiments, the transfusion-ready plasma lacks or substantially lacks a transfusion-preventing particle comprising an aspect ratio of length to width of at least 10, and wherein the particle is at least 65 microns in length, or at least 100 microns in length, or at least 150 microns in length, or at least 200 microns in length.

In some embodiments, the fibers, single-layer filters, or beads are coated with at least one blood group antigen.

In some embodiments, the reconstituted plasma is reconstituted from freeze dried plasma. In some embodiments, the reconstituted plasma is reconstituted from spray-dried plasma.

In some embodiments, the filter is configured for attachment (e.g., inline attachment) in a blood product collection or storage device. In some embodiments, the filter is configured with a Luer lock fitting part.

In some embodiments, the filter lacks or substantially lacks pores defining a lumen having a diameter larger than 50 microns along a straight path through the filter, or lacks or substantially lacks pores defining a lumen having a diameter larger than 25 microns along a straight path through the filter, or lacks or substantially lacks pores defining a lumen having a diameter larger than 10 microns along a straight path through the filter.

In some embodiments, the straight path through the filter is less than about 10 centimeters in length, or is less than about 5 centimeters in length, or is less than about 1 centimeter in length, or is less than about 500 millimeters in length, or is less than about 100 millimeters in length, or is less than about is less than about 50 millimeters in length, or is less than about 1 millimeter centimeter in length, or is less than about 500 microns in length or is less than about 100 microns in length, or is less than about 50 microns in length, or is less than about 10 microns in length.

### BRIEF DESCRIPTION OF THE DRAWINGS

FIG. 1 is a diagram showing a high resolution view of the randomly stacked arrangement of individual fibers in a non-limiting tortuous path filter of the invention.

FIGS. 2A and 2B are diagrams showing the surface (FIG. 2A) and cross-section (FIG. 2B) of a non-limiting single layer filter having a plain weave that can be used in a non-limiting tortuous path filter of the invention.

FIGS. 3A and 3B are diagrams showing the surface (FIG. 3A) and cross-section (FIG. 3B) of a non-limiting single layer filter having a twilled weave that can be used in a non-limiting tortuous path filter of the invention.

FIGS. 4A and 4B are diagrams showing the surface (FIG. 4A) and cross-section (FIG. 4B) of a non-limiting single layer filter having a plain dutch weave that can be used in anon-limiting tortuous path filter of the invention.

FIGS. 5A and 5B are diagrams showing the surface (FIG. 5A) and cross-section (FIG. 5B) of a non-limiting single layer filter having a twilled dutch weave that can be used in anon-limiting tortuous path filter of the invention.

FIGS. 6A and 6B are diagrams showing the surface (FIG. 6A) and cross-section (FIG. 6B) of a non-limiting single layer filter having a reverse dutch weave that can be used in anon-limiting tortuous path filter of the invention.

FIG. 7 is a disk shaped filter housing for housing (e.g., containing) a tortuous path filter of the invention, where the filter housing comprises an inline inlet port and inline outlet port.

FIG. 8 is non-limiting device that incorporates a filter housing that houses anon-limiting tortuous path filter of the invention inline with tubing as well as an inlet port and an outlet port.

FIG. 9 is non-limiting device that incorporates a drip chamber of a blood transfusion set for a filter housing that houses a non-limiting tortuous path filter of the invention, where the device further comprises roller clamp to control the flow rate of the transfusion-ready reconstituted plasma as is flow out of the tortuous path filter and into the needle, and thus intravenously enough a patient in need.

FIG. 10 is non-limiting transfusion set that incorporates the device of FIG. 8 and a blood collection bag (or simply blood bag).

### DETAILED DESCRIPTION

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Transfusion-ready reconstituted plasma is needed whenever there is the risk of traumatic injury or where patients have a need for plasma transfusion. Often, this need occurs in places where there is not a freezer for storing frozen plasma, nor a 37° C. water bath for thawing the frozen plasma. Additionally, in some embodiments, transfusion to a patient in need of transfusion-ready plasma may be preferred sooner after injury than the approximate 25 to 45 minutes required to thaw frozen plasma in a 37° C. water bath.

Since the major component of plasma is water, plasma can be dried and stored at ambient temperature (e.g., room temperature) and reconstituted when needed since reconstitution can be accomplished in about 10 minutes. Plasma can be spray-dried or freeze dried according to well known techniques, and reconstitution methods for reconstituting dried plasma is also well known. See, for example, Pusateri AE, Given MB, Schreiber MA, et al.: “Dried plasma: State of the science and recent developments”, Transfusion 56: S128-39, 2016; and U.S. Pat. Nos. 8,449,520; 8,533,972; 8,601,712; 9,867,782; 10,251,911; and 10,377,520, the entire contents of each of which are hereby incorporated by reference).

However, reconstituted plasma has some disadvantages that make it not transfusion-ready (e.g., not ready to be transfused into a patient in need). For example, in reconstituted plasma, particularly plasma reconstituted from dried plasma that is dried by the freeze-drying process, microscopic thin, substantially straight long fibers, referred to as ‘needles’ have been reported. Such needles are very small in size, approximately 1-9 microns (μm) in diameter. While such needles are long enough that they would be able to be trapped by the standard filters (of between about 170 to about 260 microns) included in standard blood infusion sets, at least some of the needles might realign with the fluid flow of the plasma and thereby enter the filter straight-on, much as a sewing-needle enters a fabric. In this manner, the needles may be passed through the pores of the standard filter and the resulting reconstituted plasma, despite being filtered through the filter included in a standard blood infusion set, remains not transfusion-ready. Such needles are also referred to herein as “transfusion-preventing particles.”

By a “transfusion-preventing particle” is meant a fiber or needle-shaped object with an aspect ratio of length to width of at least 10, and where the length of the particle is at least 40 microns. In some embodiments, the particle has an aspect ratio of length to width of at least 10, and where the length of the particle is at least 65 microns. In some embodiments, the particle has an aspect ratio of length to width of at least 10, and where the length of the particle is at least 100 microns. In some embodiments, the particle has an aspect ratio of length to width of at least 10, and where the length of the particle is at least 100 microns. In some embodiments, the particle has an aspect ratio of length to width of at least 10, and where the length of the particle is at least 150 microns. In some embodiments, the particle has an aspect ratio of length to width of at least 10, and where the length of the particle is at least 200 microns. In some embodiments, the aspect ratio of length to width of a particle is at least 15, or at least 20, or at least 30, or at least 50. For example, a 100 micron long particle with an aspect ratio of length to width of 25 has a width of 4 micron, and such a particle may therefore be needle-like in appearance.

Detection of a transfusion-preventing particle can be made by standard methods. For example, a light obscuration method can also be used to determine if the reconstituted plasma passed through a tortuous path filter as described herein contains any transfusion-preventing particles. The light obscuration method, which uses laser diffraction technology in a liquid particle counter, is an automated method that determines the size of the particles present in the extraction fluid and the number of each particle of at that size. A laser diode in the particle counter is directed at the liquid sample, and as particles present in the sample flow through the sensor, they diffract or interrupt the laser diode. This produces a pulse for each particle, and the amplitude of the pulse is proportional to the size of the particle.

In one non-limiting aspect, the invention provides a tortuous path filter for filtering reconstituted plasma to produce transfusion-ready plasma.

As used herein, by “tortuous path filter” is meant a filter or a media comprising at least two layers of fiber, beads, or other matrix, where the pores created by the layers do not align such that a transfusion-preventing particle small enough to pass through the first layer in one dimension cannot also pass, without turning or changing direction, through the second layer.

As the terms are used herein, by “pore” is meant a hole in a single layer of the tortuous path filter described herein. Multiple layers (e.g., multiple layers of beads, fibers, or single-layer filters), each containing pores, will have some pores in each layer that align in the multiple layers to create a lumen or a tunnel through the tortuous path filter. By “diameter” of the lumen meant the average diameter of the lumen, since the lumen is not expected to be circular. In accordance with the invention, the lumen will bend in the tortuous path filter such that the diameter of the lumen along a straight path through the tortuous path filter is small (e.g., the diameter of the lumen along a straight path is smaller than about 100 microns, or is smaller than about 50 microns, or is smaller than about 25 microns, or is smaller than about 10 microns).

In some embodiments, the straight path is less than about 10 centimeters, or less than about 5 centimeters, or less than about 1 centimeter, or less than about 0.5 centimeters, or less than about 0.25 centimeters, or less than about 100 millimeters, or less than about 50 millimeters, or less than about 25 millimeters, or less than about 10 millimeters, or less than about 5 millimeters, or less than about 2.5 millimeters, or less than about 1 millimeter, or less than about 500 micrometers (microns), or less than about 250 micrometers, or less than about 100 micrometers, or less than about 50 micrometers, or less than about 25 micrometers, or less than about 10 micrometers in length.

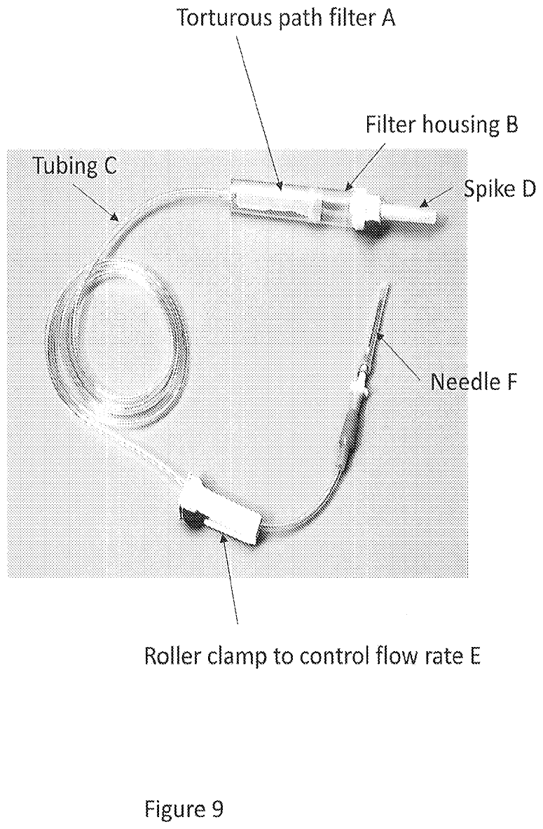

It will be understood that in some embodiments, the thickness of the filter described herein may be larger than the straight path. Rather, the straight path is merely the path along which a lumen having a small diameter (e.g., a diameter less than 10 microns) extends. For example, the filter may be 2 centimeters thick (see, for example, the torturous path filter A in FIG. 9), where the thickness is the measured as the amount of filter inline with the tubing C of FIG. 9, but the straight path may be, for example, less than 2 centimeters. For example, a straight path in torturous path filter A of FIG. 9 may be less than 250 microns, as a lumen twists and turns through the thickness of the torturous path filter A of FIG. 9. Indeed, a single lumen in a torturous path filter may comprise multiple straight paths, of various lengths, each with a diameter along each of the straight paths of less than about 100 microns.

In some embodiments, the tortuous path filter of the invention meets the United States Pharmacopeia (USP) 788 standard. USP 788 “Particulate Matter in Injections” provides the pharmaceutical industry with guidance on the quantification of particulate matter in injectable finished dosage forms. The test procedure per USP 788 allows for enumeration of particles by light obscuration or microscopic particle examination and limits are provided in the standard for both small volume (less than or equal to 100 mL) and large volume (more than 100 mL) injections for each examination method for particles greater than and equal to 10 microns and 25 microns. To meet the USP 788 standard, the device shall provide no more than the following effluent levels when subjected to filtered water or other solvent flush test: in a dosage equal to that being administered (e.g., a unit of reconstituted plasma), no more than 12 particles/mL that are greater than or equal to 10 μm and no more than 2 particles per mL that are greater than or equal to 25 microns.

In some embodiments, the tortuous path filter of the invention meets the American National Standards Institute (ANSI)/Association for the Advancement of Medical Instrumentation (AAMI) BF7 standard, as described in the American National Standard for Blood Transfusion Micro-Filters, ANSI/AAMI BF7 published by the Association for the Advancement of Medical Instrumentation, 1989. Per the ANSI/AAMI BF7 standard, the device shall provide no more than the following effluent particle levels when subjected to water or other solvent flush test: 1) 0.90 particle per mL of solvent flush larger than 10 microns; 2) 0.35 particle per mL of solvent flush larger than 25 microns; 0.65 fiber per mL or solvent flush.

In some embodiments, the tortuous path filter of the invention meets the American National Standards Institute (ANSI)/Association for the Advancement of Medical Instrumentation (AAMI) BF7 standard and also meets the USP 788 standard.

The ANSI/AAMI BF7 and the USP 788 standards are based on particulate matter quantitation analysis for transfusion devices and injections, respectively. The ANSI/AAMI BF7 test standard has specifications for particulate and fibers whereas USP 788 only has particulate counts. For, example, to determine if a device meets the test requirements, fluid (e.g., filtered water or other solvent, as applicable by the standard) is passed through the device (e.g., a tortuous path filter described herein) is then passed through a membrane filter of less than or equal to 1.0 micron (0.8 microns for ANSI/AAMI BF7 test). After drying, the membrane filter is then subjected to a microscopic particulate enumeration for particles greater than or equal to 10 microns and particles greater than or equal to 25 microns particle sizes as well as fibers.

Of course, the skilled person will understand that any method may be utilized for determining if a tortuous path filter described herein meets the ANSI/AAMI BF7 and/or USP 788 standard.

In some embodiments, a tortuous path filter completely or substantially lacks pores that create a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) having a lumen wider than about 100 microns (about 100 μm) in diameter along the length of the straight path through the filter. In some embodiments, a tortuous path filter lacks or substantially lacks pores that create a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns or less than about 50 microns, or less than about 25 microns, or less than about 10 microns in length) having a lumen wider than about 50 microns (about 50 μm) in diameter along the length of the straight path through the filter. In some embodiments, a tortuous path filter lacks or substantially lacks pores that create a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) having a lumen wider than about 40 μm (about 40 microns) in diameter along the length of the straight path through the filter. In some embodiments, a tortuous path filter lacks or substantially lacks pores that create a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) having a lumen wider than about 35 microns (about 35 μm) in diameter along the length of the straight path through the filter. In some embodiments, a tortuous path filter lacks or substantially lacks pores that create a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) having a lumen wider than about 150 microns (about 150 μm) in diameter along the length of the straight path through the filter.

In various non-limiting embodiments, the tortuous path filter may comprise beads that are not regularly aligned such that the filter lacks or substantially lacks any pores having a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) with a lumen consistently wider than, for example, about 100 microns (about 100 μm) in diameter along the length of the straight path through the beads in the filter. In another non-limiting example, the tortuous path filter comprises various fibers stacked upon each other in a random manner (nonwoven filter media), such that the filter lacks or substantially lacks any pores having straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) with a lumen consistently wider than, for example, about 100 microns (about 100 μm) in diameter along the length of the straight path through the randomly stacked fibers in the filter. In yet another non-limiting example, the tortuous path filter may comprise multiple layers of single-layer filters that are stacked so that the pores of one filter layer do not align with the pores of the other layers such that the filter lacks or substantially lacks any pores having a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) with a lumen consistently wider than, for example, about 100 microns (about 100 μm) in diameter is created through the multiple layers of filters.

As used herein, by “substantially lacks” with reference to a torturous path filter and pores, is meant that the tortuous path filter has fewer than 20% of its pores, or fewer than 10% of its pores, or fewer than 5% of its pores, or fewer than 1% of its pores having straight path with a lumen consistently wider than the indicated diameter (e.g., about 100 microns) along the length of the straight path. For example, where the indicated diameter is about 100 microns, a tortuous path filter is said to substantially lack pores that create a straight path having a lumen wider than about 100 microns in diameter along the length of the straight path through the filter when fewer than 20%, or fewer than 10%, or fewer than 5% or fewer than 1% of the all the pores in the tortuous path filter creates a straight path through the filter where the lumen of the path is wider than about 100 microns in diameter along the length of the straight path. In some embodiments, the straight path is less than about 10 centimeters, or less than about 5 centimeters, or less than about 1 centimeter, or less than about 0.5 centimeters, or less than about 0.25 centimeters, or less than about 100 millimeters, or less than about 50 millimeters, or less than about 25 millimeters, or less than about 10 millimeters, or less than about 5 millimeters, or less than about 2.5 millimeters, or less than about 1 millimeter, or less than about 500 micrometers, or less than about 250 micrometers, or less than about 100 micrometers, or less than about 50 micrometers, or less than about 25 micrometers, or less than about 10 micrometers in length.

In some embodiments, regardless of what the tortuous path filter is made of (e.g., multiple layers of single-layer filters, beads, or stacked single fibers), the tortuous path filter lacks or substantially lacks pores having a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) through the filter with a lumen of less than about 25 microns in diameter along the length of the straight path. In some embodiments, regardless of what the tortuous path filter is made of, the tortuous path filter lacks or substantially lacks pores having a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) through the filter with a lumen of less than about 10 microns in diameter along the length of the straight paths. In some embodiments, regardless of what the tortuous path filter is made of, the tortuous path filter lacks or substantially lacks pores having a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) through the filter with a lumen of less than 100 microns in diameter along the length of the straight path. In some embodiments, regardless of what the tortuous path filter is made of, the tortuous path filter lacks or substantially lacks pores having a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) through the filter with a lumen of less than 150 microns in diameter along the length of the straight path. In some embodiments, regardless of what the tortuous path filter is made of, the tortuous path filter lacks or substantially lacks pores having a straight path (e.g., a straight path that is less than about 10 cm, or is less than about 5 cm, or is less than about 1 cm, or less than about 0.5 cm, or less than about 0.25 cm, or less than about 0.1 cm, or less than about 50 mm, or less than about 25 mm, or less than about 10 mm, or less than about 5 mm, or less than about 2.5 mm, or less than about 1 mm, or less than about 500 microns, or less than about 250 microns, or less than about 100 microns, or less than about 50 microns, or less than about 25 microns, or less than about 10 microns in length) through the filter with a lumen of less than 200 microns in diameter along the length of the straight path.

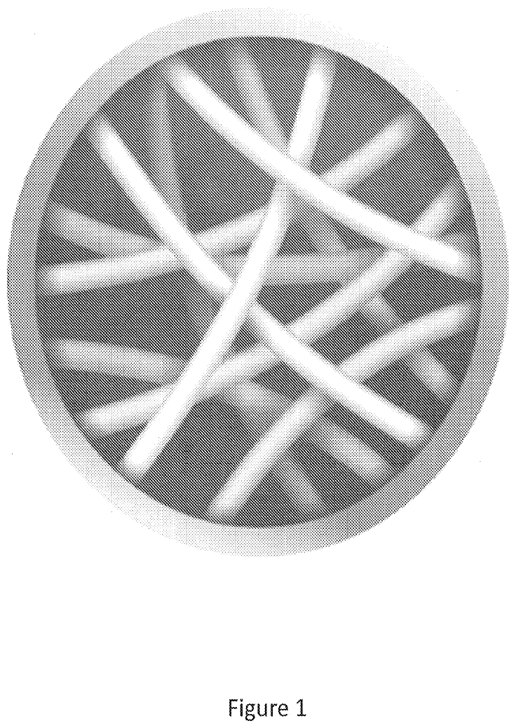

In some embodiments, the tortuous path filter as described herein may comprise multiple layers of individual fibers, where the fibers do not align atop one another. As a result, the tortuous path filter lacks a substantial number of pores that maintain their diameter through the depth of the filter. The tortuous path filter can be constructed, for example, by the stacking of fibers in an unorganized manner, such as is shown in FIG. 1. As FIG. 1 shows at a microscopic level, none of the paths traversing the filter forms a straight line through the filter. No straight path through the tortuous path filter of the non-limiting torturous path filter shown in FIG. 1 has a lumen larger than 40 microns in diameter along the length of the straight path.

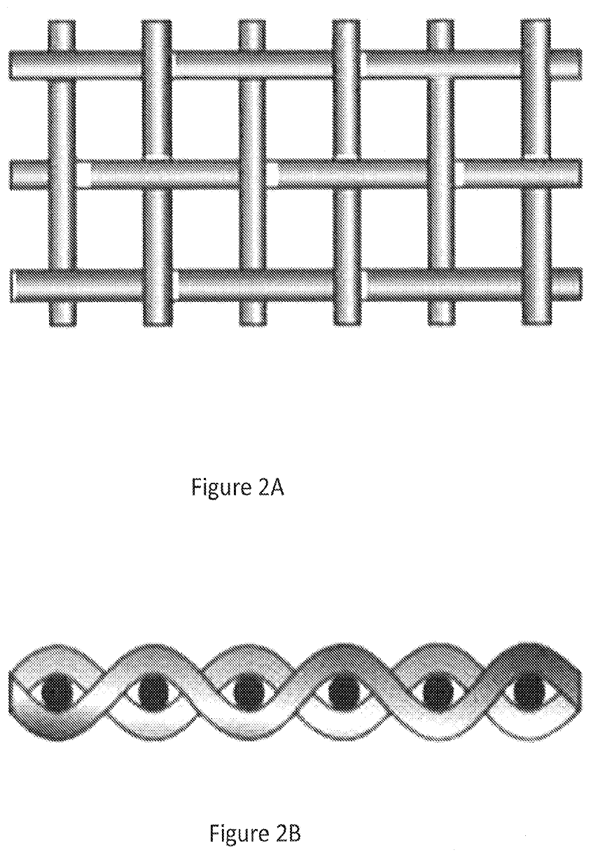

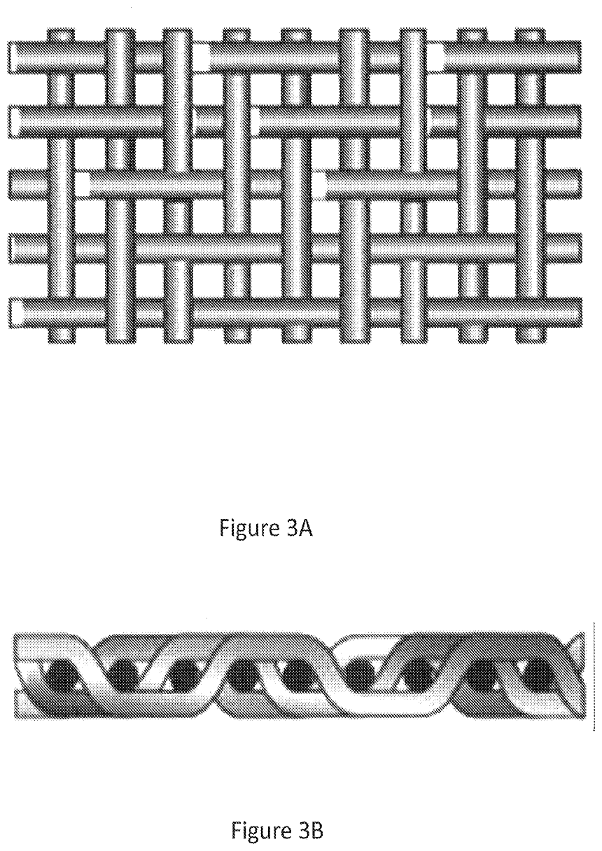

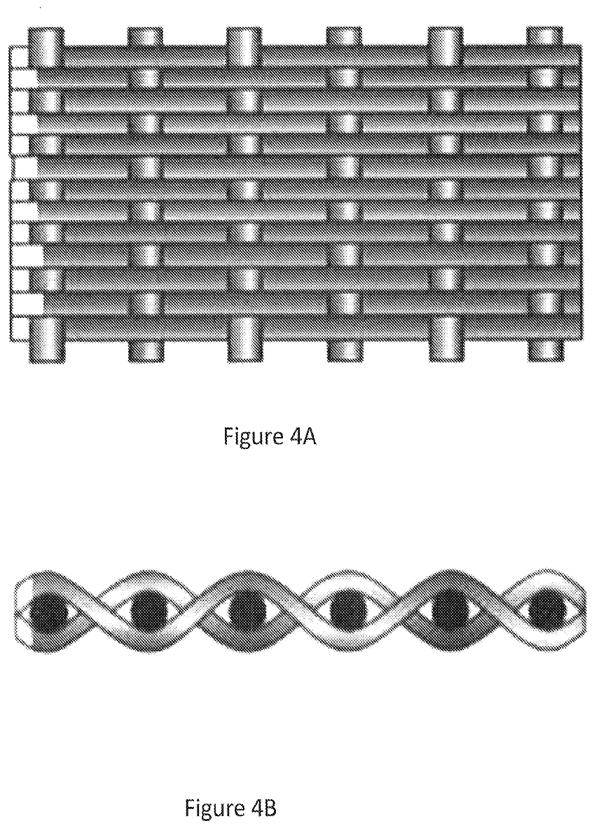

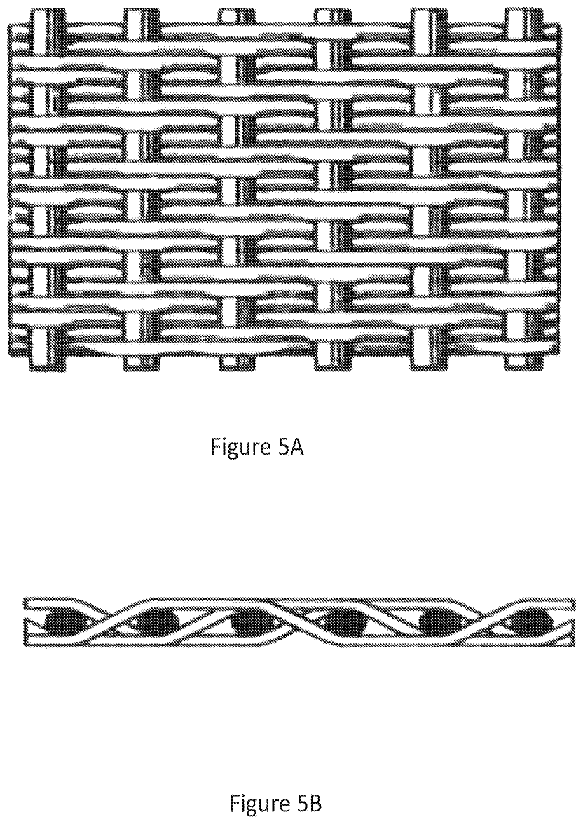

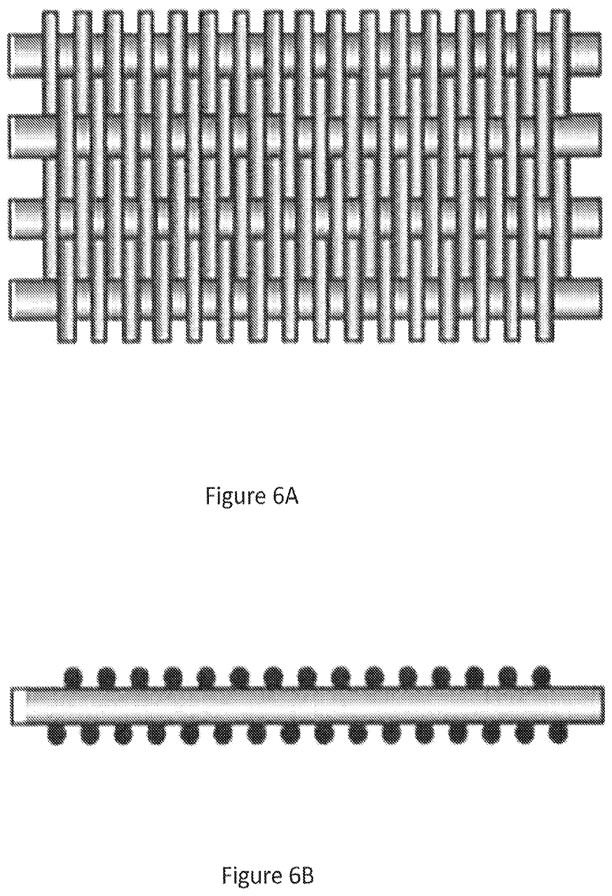

In some embodiments, the tortuous path filter as described herein comprises multiple layers of fibers that are woven into a single-layer filter. In some embodiments, the single-layer filter has uniform-sized pores. This can be achieved, for example, by weaving strands of individual fibers of approximately the same diameter. Non-limiting examples of filters with uniform pores are filters woven by plain weave (FIGS. 2A-2B) and twilled weave (FIGS. 3A-3B). In some embodiments, the single layer filter has pores of different sizes. This can be achieved, for example, by weaving strands of individual fibers with different diameters. For example, in dutch weaving, the warp fibers have a different size diameter than the weft fibers. In some embodiments, the warp fibers have a larger diameter than the weft fibers. In some embodiments, the warp fibers have a smaller diameter than the weft fibers. Non-limiting examples of filters with non-uniform sized pores are filters woven by various dutch weaves, such as plain dutch weave (FIGS. 4A-4B), twilled dutch weave (FIGS. 5A-5B), and reverse dutch weave (FIGS. 6A-6B).

It will be understood, of course, that multiple weaving methods may be employed with various materials being used for the weft and warp fibers to create a single layer filter that can then be stacked with other single layer filters to create a non-limiting example of a tortuous path filter. It will be understood that in this embodiment, the warp and weft fibers may be of different material, dissimilar size, diameter or profile (e.g., round, irregular such as trilobal, oval, etc.) to create a single layer filter. Each of the woven layers may be stacked upon one another. In some embodiments, for example, where the pore size in the single layer is not uniform, the woven layers can be stacked directly on each other. In some embodiments, the single layers are stacked in an unorganized manner so that the gaps or pores in each of the single layer filter does not completely align with the gaps or pore in the single layer. In some embodiments, fewer than 85%, or fewer than 90% of the pores, or fewer than 95%, in the multiple layers do not maintain their diameter along a straight path throughout the depth of the filter.

In some embodiments, the average diameter of the pores along a straight path through the depth of the tortuous path filter is smaller than 40 microns. In some embodiments, at least 90%, or at least 95% of the pores has a diameter smaller than 40 microns along a straight path through the depth of the tortuous path filter. In some embodiments, the average diameter of the pores along a straight path through the depth of the tortuous path filter is smaller than 25 microns. In some embodiments, at least 90% or at least 95% of the pores has a diameter smaller than 25 microns along a straight path through the depth of the tortuous path filter. In some embodiments, the average diameter of the pores along a straight path through the depth of the tortuous path filter is smaller than 10 microns. In some embodiments, at least 90% or at least 95% of the pores has a diameter smaller than 10 microns along a straight path through the depth of the tortuous path filter.

In some embodiments, the tortuous path filter described herein is placed in an inline filter housing with inlet and outlet ports such as a disk filter. By “inline” is simply meant that a filter is connected to a tubing or other device (e.g., a needle) such that a fluid (e.g., reconstituted plasma) can flow through the filter directly into the inline connected device. In some embodiments, the fluid flowing from one device (e.g., a filter) into an inline device (e.g., tubing) remains sterile. In some embodiments, the fluid flowing from one device (e.g., a filter) into an inline device (e.g., tubing) does not leak out of the connecting point. Inline connections are well known, and in some embodiments, a tortuous path filter described herein is configured for inline attachment to tubing and other devices in, for example, a blood collection system. For example, a tortuous path filter described herein may be configured to comprise a male or female Luer lock fitting part. The Luer lock (or Luer taper) is a standardized system of small-scale fluid fittings used for making leak-free connections between a male-taper fitting and its mating female fitting part on medical and laboratory instruments, including hypodermic syringe tips and needles or stopcocks and needles.

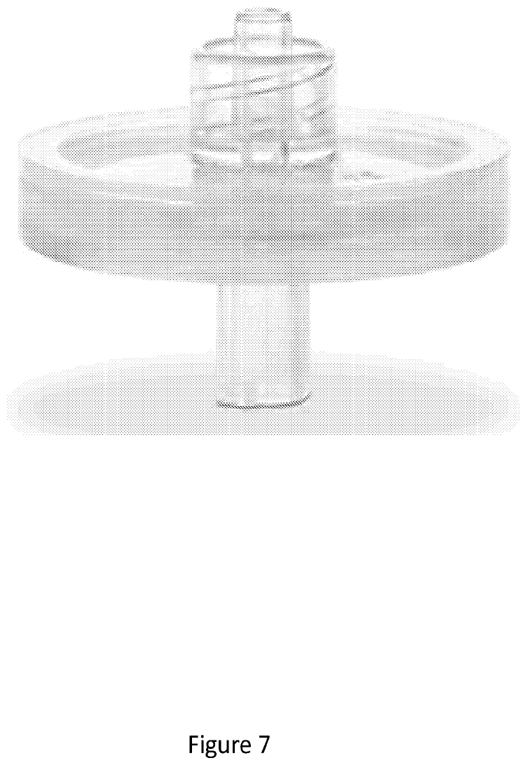

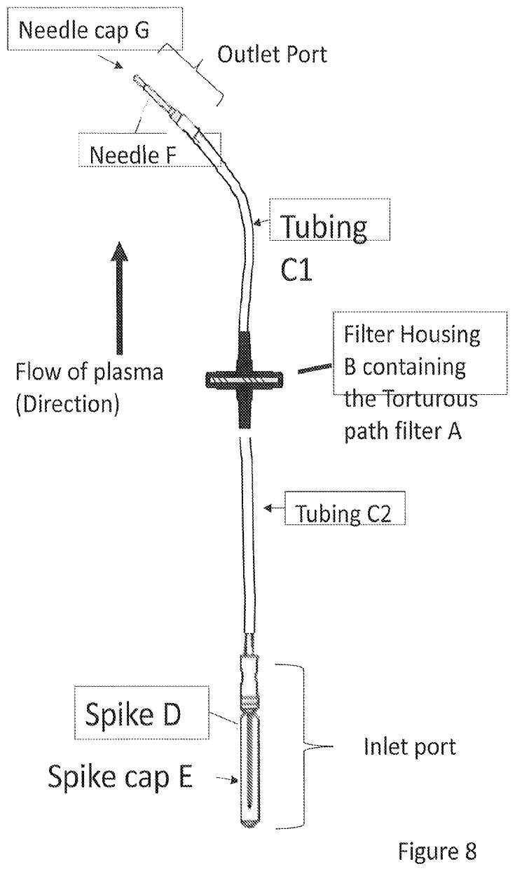

FIG. 7 shows a filter housing with an inlet and outlet port that can be connected inline via tubing to a spike for insertion into a blood bag and/or to a needle for insertion into a patient. FIG. 8 shows a non-limiting example of tortuous path filter within an inline disk filter housing with an inlet port (with spike) and outlet port (with needle, such as a 16 gauge needle) for intravenous injection into the patient in need to be transfused. Specifically, the device of FIG. 8 comprises a tortuous path filter A within an inline disk filter housing B with an inlet port and an outlet port, the inlet port being connected to tubing (C2) with a spike (D) and spike cap (E), and the outport being connected to tubing (C1) with a needle (e.g., a 16 gauge needle shown as F) and a needle cap (G).

Other filter configurations are within the scope of the present invention including but not limited to pleated filters, packed columns, capsules, etc. For example, FIG. 9 is yet another non-limiting example of the device of the invention. In FIG. 9, a drip chamber of a blood transfusion set acts as the filter housing B, and the tortuous path filter A (e.g., comprised of multiple layers of dutch woven single layers, such as those shown in FIGS. 2A-B and 3A-B) contained within the housing. In this example, the inlet port of the device includes the spike (D) which is directly connected to the inlet port of the filter housing. The outlet port of the filter housing is connected inline via tubing C to a needle F. The flow of the plasma through the tubing into the needle (and thus intravenously into a patient in need) can be controlled by the roller clamp (E in FIG. 9) in this non-limiting example of the device of the invention.

In some embodiments, the tortuous path filter described herein comprises disk filters and depth filters with low hold up volumes to reduce waste.

By “reconstitute” is meant the process of combining a liquid (e.g., sterile water) and dried plasma and mixing the combination to produce reconstituted plasma. It will be understood that other liquids besides sterile water can be added to dried plasma to produce reconstituted plasma. Some non-limiting examples of liquids that can be added to dried plasma to produce reconstituted plasma include sterile saline (e.g., 09% w/v NaCl), sterile Ringer's solution, sterile phosphate buffered saline, and sterile Lactated Ringer's solution. In some embodiments, the liquid added to dried plasma to produce reconstituted plasma is selected to mitigate the risks of infusing patients with sterile water.

By “patient in need” is simply a human patient who is in need of transfusion with plasma. The patient may be a trauma patient, such as a victim of a gun or knife attack, a patient injured in battle, or a patient who has been injured in an accident (e.g., a car crash or weather-related event). The invention also contemplates patients in need who are injured at a sporting event (e.g., a player in a profession American football team), or who taking blood thinners (e.g., warfarin) and have bleeding in the brain. In other words, patients who suffer internal bleeding are also included as patients in need, as the phrase is used herein.

By “transfusion-ready” is meant that the indicated blood product (e.g., reconstituted plasma) is ready to be transfused to a patient in need. In some embodiments, the transfusion-ready reconstituted plasma lacks or substantially lacks any transfusion-preventing particles. er USP 788 Microscopic Particulate test. In some embodiments, the transfusion-ready reconstituted plasma described herein passes the United States Pharmacopeia (USP) Method <788>standard for injectable solutions. In some embodiments, the transfusion-ready reconstituted plasma meets (e.g., passes) the USP 788 Microscopic Particulate count test. And this must also be applied to reconstituted plasma. Although the needles may be safe, its removal is a non-limiting object of this invention. Limit on particle sizes under USP are defined by particle sizes of greater than 10 and 25 microns for small (e.g., 100 mL or less) and large volume (greater than 100 mL) injections. There are different methods for meeting the 788 standard.

In a first method, a solution (e.g., transfusion-ready reconstituted plasma is mixed by inverting 20 times, allowing the solutions to degas, then analyzing on a liquid particle counter. The particle counter withdraws not less than three aliquots, of not less than 5 mL. The liquid particle counter is capable of calculating the average cumulative counts, average differential counts, average cumulative counts per mL, and average differential counts per mL. Per the USP, it will automatically omit the data from the first run. The size ranges used for USP testing are greater than or equal to 10 μm (microns) and greater than or equal to 25 μm (microns). The liquid particle counter is capable of sizing and counting particles ranging from 2 μm (microns) to 100 μm (microns).

In a second method for USP 788 standard testing, microscopic testing is used. A solution (e.g., transfusion-ready reconstituted plasma) is filtered through a pre-cleaned, 1.0 μm or finer pore size, membrane filter. After filtration, the membrane filters are placed into petri slides and dried. Microscope slides are analyzed under a microscope, using a calibrated graticule, at 100× magnification. Using oblique illumination at an angle of 10° to 20°, the particles on the surface of the membrane are sized and counted using the graticule micrometer. The size ranges used for USP testing are greater than or equal to 10 μm (microns) and greater than or equal to 25 μm (microns). The liquid particle counter is capable of sizing and counting particles ranging from 2 μm (microns) to 100 μm (microns).

As used herein, by “substantially lacks” with reference to transfusion-ready plasma (also referred to as transfusion-ready reconstituted plasma) and transfusion-preventing particles, is meant that the transfusion-ready plasma has fewer than 20% of transfusion-preventing particles, or fewer than 10% of transfusion-preventing particles, or fewer than 5% of transfusion-preventing particles, or fewer than 1% of transfusion-preventing particles as compared to the total of particles in the tested aliquot. For example, where a 10 mL aliquot of plasma is tested and 100 particles are detected, a transfusion-ready plasma is said to substantially lack transfusion-preventing particle when fewer than 20%, or fewer than 10%, or fewer than 5% or fewer than 1% of the all the particles in that 10 mL aliquot have an aspect ratio of length to width of at least 10, and where the length of the particle is at least 40 microns.

In some embodiments, the transfusion-ready reconstituted plasma is produced by passing reconstituted plasma that is not transfusion ready through a tortuous path filter as described herein, wherein the filtered reconstituted plasma is referred to as “transfusion-ready reconstituted plasma”. In some embodiments, the transfusion-ready reconstituted plasma lacks or substantially lacks one or more transfusion-preventing particles.

In some embodiments, the transfusion-ready reconstituted plasma comprises less than 50% of the blood group antibody as compared to reconstituted plasma that is not transfusion ready. In some embodiments, the transfusion-ready reconstituted plasma comprises less than 30% of the blood group antibody as compared to reconstituted plasma that is not transfusion ready. In some embodiments, the transfusion-ready reconstituted plasma comprises less than 20% of the blood group antibody as compared to reconstituted plasma that is not transfusion ready.

In some embodiments, the transfusion-ready reconstituted plasma comprises less than 50% of particles longer than 5 μm (also written as 5 μm, 5 microns or 5 micrometers) in length as compared to reconstituted plasma that is not transfusion ready. In some embodiments, the transfusion-ready reconstituted plasma comprises less than 30% of particles longer than 5 μmin length as compared to reconstituted plasms that is not transfusion ready. In some embodiments, the transfusion-ready reconstituted plasma comprises less than 20% of particles longer than 5 μm in length as compared to reconstituted plasms that is not transfusion ready.

Another feature of plasma that is used to transfuse human patients may be the removal from the plasma of antibodies that react against the ABO antigen that is expressed by the recipient's blood cells. For example, if the person donating plasma is from the blood group B, his blood cells have B antigens on their surface, and his plasma has anti-A antibodies (but not anti-B antibodies). A donor with A type blood has anti-B antibodies in his plasma, but will not have anti-A antibodies in his plasma. A donor with the O blood group (reflecting the majority of the US population) has anti-A antibodies as well as anti-B antibodies in his plasma. Another blood group antigen is the Rh factor-donors who are Rh negative will have anti-Rh antibody in their plasma. These blood group antibodies persist when the plasma is dried, and also remain present when the dried plasma is reconstituted.

By “blood group antibody” means an antibody that specifically binds to a blood group antigen. The A antigen, the B antigen, and the Rh factor antigen are collectively referred to as “blood group antigens”. Blood group antigens are either sugars or proteins, and they are attached to various components in the red blood cell membrane. For example, the antigens of the ABO blood group are sugars. They are produced by a series of reactions in which enzymes catalyze the transfer of sugar units. A person's DNA determines the type of enzymes they have, and, therefore, the type of sugar antigens that end up on their red blood cells.

The structures of the blood group A antigen and the blood group B antigen are shown below.

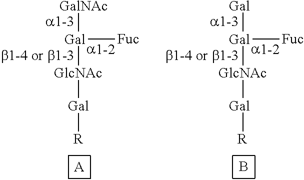

These antigens can be synthesized, for example, according to standard methods, and used to coat fibers, beads, or filters of various embodiments of the tortuous path filter in accordance with the invention. Alternatively or additionally, the A and/or B blood group antigens can be coated onto a blood bag, such as the blood collection bag into which donor plasma is collected prior to being dried, or the blood collection bag in which dried plasma is mixed with a liquid to produce reconstituted plasma, or in blood collection bag reconstituted plasma flows into after passing through a tortuous path filter as described herein. Blood type antibodies will bind to the antigen coated onto the filter or the bag and thus be removed from the plasma prior to its transfusion into a patient in need.

The Rh antigen can be similarly used to coat the materials (e.g., beads, fibers, or filter layers) of the tortuous path filter in various embodiments of the invention, or to coat the inner layer of a blood collection bag.

Accordingly, in some embodiments, some or all of the fibers in the tortuous path filter are coated with blood group antigen. For example, in some embodiments, a fiber in the tortuous path filter is coated with blood group antigen A. In some embodiments, a fiber in the tortuous path filter is coated with blood group antigen B. In some embodiments, a fiber in the tortuous path filter is coated with blood group antigen A and blood group antigen B. Methods for coating surfaces with blood group antigens are well known. For example, blood typing kits have been commercially available for decades.

In another embodiment, the transfusion-ready plasma described herein is devoid of or substantially devoid of blood group antibodies. In some embodiments, the blood group antibody specifically binds to the A blood group antigen. In some embodiments, the blood group antibody specifically binds to the B blood group antigen. In some embodiments, the blood group antibody specifically binds to the Rh factor blood group antigen. For example, the transfusion-ready plasma is devoid of or substantially devoid of anti-A antibodies; the transfusion-ready plasma is devoid of or substantially devoid of anti-B antibodies, and/or the transfusion-ready plasma is devoid of or substantially devoid of both anti-A and anti-B antibodies). In some embodiments, the transfusion-ready plasma is devoid of or substantially devoid of anti-Rh factor antibodies.

As used herein, by “substantially devoid of” means that at least 80% or at least 90% or at least 95% or at least 98% of the intended substance (in this case blood group antibodies) is removed from the plasma as compared to the amount of that blood group antibody in the plasma prior to removing the indicated blood group antibody. In some embodiments, “substantial devoid of” includes low titer plasma, low titer group O whole blood (LTOWB), as well as plasma containing low levels of antibodies making it safe for transfusion with any blood type. “LTOWB” is blood/plasma with low amounts of IgM and/or Anti-A and Anti-B antibodies. For example, plasma donated by a donor having blood group O, where the donor has never received a blood transfusion, is LTOWB, because that donor would likely have not been exposed to either blood group antigens A or B, and thus would have a low titer (e.g., low amount) of antibodies that are anti-A or anti-B. In some embodiments, LTOWB plasma has IgM anti-A and anti-B antibodies of less than 256. In some embodiments, LTOWB plasma has IgM anti-A and anti-B of less than 150. In some embodiments, LTOWB plasma has IgM anti-A and anti-B of less than 50.

Thus, in some embodiments, the removal or absence of these blood group antibodies can be an additional feature of an embodiment of a tortuous path filter described herein, particularly if the plasma donor does not have the blood group O. For example, if the tortuous path filter is comprised of randomly stacked beads, the beads can be coated with a blood group antigen of choice (e.g., coated with A antigen) such that anti-A antibodies in the plasma will bind to the beads and not pass through the filter. Additionally and/or alternatively, a tortuous path filter as described herein may be included in a plasma collection system that has a feature where anti-A and/or anti-B and/or anti-Rh factor antibodies are removed from the reconstituted plasma. Where the collection system comprises both a tortuous path filter and a separate antibody-removing component (e.g., a filter or storage in an A and/or B and/or Rh factor antigen-coated bag), the reconstituted plasma may have the antibodies removed before passage through the tortuous path filter, after passage through the tortuous path filter, or at the same time as the plasma passed through the tortuous path filter.

It will be understood that the various embodiments of the tortuous path filter described herein may be included in a plasma collection system that has a feature where any other antibody of interest can be specifically removed from the reconstituted plasma. For example, it may be desirable to remove antibodies that bind to antigens encoded by genes on the Y chromosome, if the donor is female and the intended recipient is male.

In some embodiments, the tortuous path filter further comprises the ability to block passage of anti-A and/or anti-B and/or anti-Rh factor antibodies. Thus, in some embodiments, the torturous path filter as described herein filters anti-A and/or anti-B and/or anti-Rh factor antibodies out of the filtered reconstituted plasma.

Accordingly, in another aspect, the invention provides a blood group antibody-reducing tortuous path filter. In some embodiments, passage of reconstituted dried plasma through a blood group antibody-reducing tortuous path filter, such as the filter described herein, results in transfusion-ready plasma that also lacks anti-A and/or anti-B and/or anti-Rh factor antibodies.

Plasma comprises about 90% water. Additional components in plasma, including salts, nutrient molecules (such as amino acids), enzymes, and other proteins including antibodies, complement proteins, coagulation and clotting factors (as well as precursors thereof including fibrinogen), cytokines, hormones, and proteins such as albumin that maintain osmotic pressure of the plasma.

In some embodiments, the non-water components in reconstituted plasma can be measured to determine their presence and/or activity, and those measurements can be compared to fresh plasma.

Plasma components of reconstituted plasma can be measured using a variety of tests or assays. Some measurements that can be assessed in reconstituted plasma include, without limitation, prothrombin time (PT), international normalized ratio (INR), activated partial thromboplastin time (aPTT), activity of heat-labile proteins (e.g., Factor V, Factor VIII), activity of anticoagulant proteins (e.g., Protein S, Protein C), quantity, size, and activity of large coagulation proteins prone to aggregation and degradation (e.g., fibrinogen, von Willebrand factor), markers of coagulation activation (e.g., thrombin-antithrombin complexes, fibrin degradation products), pH, and characterization of particles in the reconstituted plasma. In various embodiments, the reconstituted plasma comprises a pH of between about 7.38 and 7.42.Fresh plasma has a known number of components and characteristics. The reference range for components and characteristics (e.g., prothrombin time) of plasma from a normal healthy person are set forth in Table 1 below.

TABLE 1

| | Standard reference | Unit of |
|---|---|---|
| Plasma component or characteristic | Range | measure |
| PT (prothrombin time) | 10.0-13.0 | Seconds |
| APTT (activated partial prothrombin time) | 23-30 | Seconds |
| Albumin | 3.5-4.5 | g/dL |
| Fibrinogen | 1.5-4.5 | g/L |
| D-Dimers (Not the DVT/PE cut off value) | 0.1-0.45 | mg/L |
| D-Dimer | Less than 500 | ng/L |
| Antithromin III | 80-120 | IU/dL |
| Protein S | 60-140 | IU/dL |
| Protein C | 70-130 | IU/dL |
| Thrombin Time | 13-20 | IU/dL |
| Lupus Anticoagulant | <1.2 | Ratio |
| Willibrand factor antigen | Approximately 100 | mg/L |
| Factor II | 50-150 | IU/dL |
| Factor V | 50-150 | IU/dL |
| Factor VII | 50-150 | IU/dL |
| Factor VIII | 50-150 | IU/dL |
| Factor IX | 50-150 | IU/dL |
| Factor IX | Approximately 4 | mg/L |
| Factor X | 50-150 | IU/dL |
| Factor X | Approximately 10 | mg/L |
| Factor XI | 50-150 | IU/dL |
| Factor XI | Approximately 7.0 | mg/L |
| Factor XII | 50-150 | IU/dL |

Fresh frozen plasma (FFP) contains normal plasma levels of all the clotting factors, albumin and immunoglobulin. A unit is typically 200-250 mL. Transfusion-ready reconstituted plasma, like fresh frozen plasma, contains normal plasma levels of all the clotting factors and albumin but contains a reduced number of immunoglobulins because of the removal of blood group antibodies.

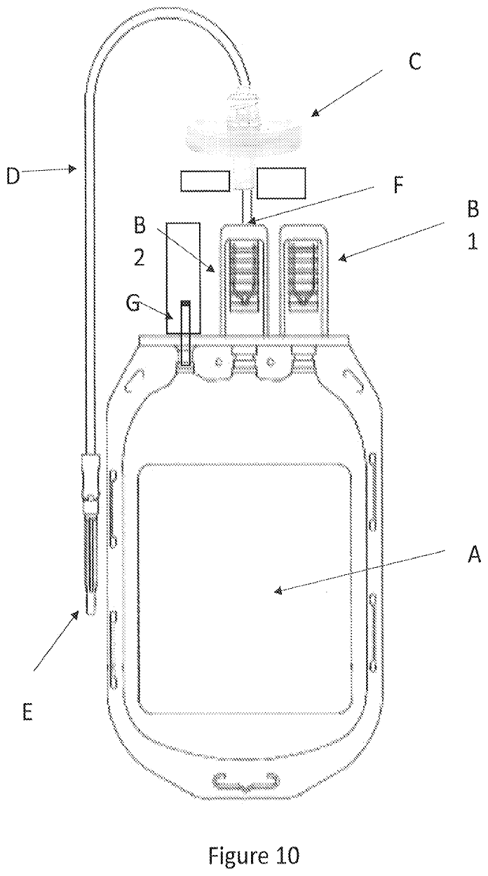

FIG. 10 shows a non-limiting example of a device of the invention. Dried plasma can be stored within compartment A (e.g., of a blood bag). FIG. 10 shows closed-off tubing part G (or, for example, a breakaway cannula) through which the donor plasma was added into the bag. In some embodiments, the donor plasma is dried prior to entry into compartment A. In some embodiments, the donor plasma is dried after entry into compartment A. In some embodiments, the interior walls of compartment A are coated with one or more blood group antigens (e.g., coated with A antigen and/or coated with B antigen and/or coated with Rh factor antigen). The reconstituting liquid (e.g., sterile water) can be added, for example, through cannula B1 when the dried plasma is reconstituted when there is a patient in need. The bag may then be mixed and, upon mixing to form reconstituted plasma, may be turned upside down such that the bag contents flow through cannula B2 into an inline a tortuous path filter within filter housing C. The transfusion-ready reconstituted plasma then flows through tube D to the needle E that is intravenously inserted into the patient in need.

It should be noted that although two cannulas (B1 and B2) are depicted in FIG. 10 for easily visualization, only one cannula or other entry port into compartment A is necessary. For example, the closed off tubing or breakaway cannula (part G in FIG. 10) may not be closed off but instead include a cannula or port that self-seals when the bag containing the dried plasma is in storage. Once reconstitution is desired, the reconstituting liquid is entered through the cannula (or port), and once the appropriate amount of liquid is added and the vehicle through which the liquid was added (e.g., a 16 gauge needle) is withdrawn, the cannula or port self-seals, enabling manual or automated mixing of the bag to fully reconstitute the dried plasma. Following full reconstitution, a spike (inserted spike shown as part D in FIG. 8) is inserted into the same cannula or port to inline connect the tortuous path filter, filter housing, tubing, and needle to the bag comprising reconstituted plasma in compartment A.

A tortuous path filter, where the filter itself is coated with blood group antigens A, B, and Rh factor, is prepared. To do this, various sizes and/or shapes (both diameter and length) and made of various materials are coated with A antigen, B antigen, or Rh factor. The fibers are then mixed together and then randomly stacked such as is shown in FIG. 1. The final matrix is then housed in a housing such as that depicted in FIG. 7.

### Example 1

### Example 2

A device such as that shown in FIG. 8 is made. A tortuous path filter is housed in a disk-shaped filter housing, where the filter housing has inline inlet and outlet ports. The ports are each attached to sterile tubing, with the free end of each tube attached to a sterile spike. A spike cap covers each of the spikes to keep them sterile during storage.

When dried plasma is reconstituted to make reconstituted plasma in a blood bag with a cannula (blood bag not shown), the spike cape (part E in FIG. 8) at the inlet port of the filter housing covering spike is removed and the spike (D in FIG. 8) is inserted into a cannula in the blood bag. The blood bag is lifted above the filter housing (B in FIG. 8) containing the tortuous path filter (part A in FIG. 8) such that the reconstituted plasma will flow through the tortuous path filter and transfusion-ready reconstituted plasma will flow out of the outlet port of the filter housing and into tubing C2. Once tubing C2 is filled with transfusion-ready reconstituted plasma, and no large air bubbles are present in the tubing C2, spike cap E2 is removed and spike D2 can be administered intravenously to a patient in need.

### Example 3

During an American football game, one of the players is injured where compound fractures occur in both the tibia and ulna in his right arm, resulting in one of the broken ends of the tibia breaking through the man's right forearm. An ambulance is immediately dispatched onto the field. The ambulance has two types of plasma-fresh frozen plasma (FFP) and freeze-dried plasma. Thawing the FFP will require at least 45 minutes. The freeze-dried plasma is stored in a device including a blood bag (part A in FIG. 10) whose internal sides are coated with blood group antigens A and B. The blood bag is equipped with two cannulas such as the bag depicted in FIG. 10. The first canula (B1 in FIG. 10) is designed to receive a spike delivering sufficient water to reconstitute the plasma. The second canula (B2 in FIG. 10) is designed to receive a spike (part F in FIG. 10) in-line with a tortuous path filter (part C in FIG. 10) through which the reconstituted plasma must pass before it is transfused into the patient. Note that in FIG. 10, part G is a closed off tubing that is closed off after the plasma (either dried or to be dried) is added to the bag, so that the dried plasma does not become unsterile during storage.

Given time considerations, the paramedics at the scene use the freeze-dried plasma. An appropriate volume of water, for example 200 mLs sterile water is added to the freeze-dried plasma bag via the first cannula (B1 in FIG. 10). The bag or device containing dried plasma is then mixed appropriately manually or with automated mixer/rocker equipment to fully reconstitute the dried plasma to make reconstituted plasma and also to allow all or the majority of the blood group antibodies in the plasma to remain bound to the internal sides of the bag, thereby removing these blood group antibodies, if present, from the reconstituted plasma. The reconstituted plasma-containing device is then hung on a blood bag rack and the needle (E in FIG. 10) at the end of the sterile tubing (part D in FIG. 10) is injected intravenously into the injured player. The reconstituted plasma drips from the bag (part A in FIG. 10) through the through the tortuous path filter (part C in FIG. 10) into tubing (D in FIG. 10) and is delivered by transfusion intravenously to the injured player via needle (E in FIG. 10).

The embodiments of the invention described above are intended to be merely exemplary; numerous variations and modifications will be apparent to those skilled in the art. All such variations and modifications are intended to be within the scope of the present invention as defined in any appended claims.

1. A tortuous path filter for filtering reconstituted plasma, the filter comprising at least two layers of fibers, single-layer filters, or beads, wherein the filter lacks or substantially lacks pores defining a lumen having a diameter larger than 100 microns along a straight path through the filter.

2. The filter of claim 1, wherein said fibers, single-layer filters, or beads are coated with at least one blood group antigen.

3. The filter of claim 1, wherein the reconstituted plasma is reconstituted from freeze dried plasma.

4. The filter of claim 1, wherein the filter is configured for attachment in a blood product collection or storage device.

5. The filter of claim 1, wherein the filter lacks or substantially lacks pores defining a lumen having a diameter larger than 50 microns along a straight path through the filter.

6. The filter of claim 1, wherein the filter lacks or substantially lacks pores defining a lumen having a diameter larger than 25 microns along a straight path through the filter.

7. The filter of claim 1, wherein the filter lacks or substantially lacks pores defining a lumen having a diameter larger than 10 microns along a straight path through the filter.

8. The filter of claim 1, wherein the straight path is less than about 10 centimeters in length, or is less than about 5 centimeters in length, or is less than about 1 centimeter in length, or is less than about 500 millimeters in length, or is less than about 100 millimeters in length, or less than about 1 millimeter in length, or is less than about 500 microns in length, or is less than about 100 microns in length, or is less than about 50 microns in length, or is less than about 10 microns in length.

9-18. (canceled)

19. The filter of claim 1, wherein the filter meets a standard selected from the group consisting of a United States Pharmacopeia (USP) 788 standard and American National Standards Institute (ANSI)/Association for the Advancement of Medical Instrumentation (AAMI) BF7 standard.

20. A medical device comprising the tortuous path filter of claim 1, said filter inline with a first tubing to an inlet port and a second tubing to an outlet port, wherein said inlet port further comprises a spike and said outlet port further comprising a needle for intravenous injection.

21. The device of claim 20, further comprising a blood bag comprising a port injectable with the spike of the tortuous path filter.

22. The device of claim 21, wherein blood bag comprises a blood bag with an internal surface, said internal surface coated with one or more blood group antigens.

23. A method for producing transfusion-ready plasma comprising:
(a) reconstituting dried plasma to produce reconstituted plasma; and
(b) passing said reconstituted plasma through the tortuous path filter of claim 1 to produce transfusion-ready plasma.

24-30. (canceled)
